(19) United States
(12) Patent Application Publication
(10) Pub. No.: US 2014/0228549 A1
(43) Pub. Date: Aug. 14, 2014

(54) PROCESS FOR SEPARATION/PURIFICATION OF BIOMOLECULES

(71) Applicants: Gerhard Schembecker, Dortmund (DE); Bernhard Burghoff, Halle (Saale) (DE); Fatma Alexia Van Winssen, Dortmund (DE)

(72) Inventors: Gerhard Schembecker, Dortmund (DE); Bernhard Burghoff, Halle (Saale) (DE); Fatma Alexia Van Winssen, Dortmund (DE)

(73) Assignee: TECHNISCHE UNIVERSITAET DORTMUND, DORTMUND (DE)

(21) Appl. No.: 14/008,569

(22) PCT Filed: Mar. 27, 2012
(86) PCT No.: PCT/EP12/55434

§ 371 Date: Sep. 30, 2013

(30) Foreign Application Priority Data
Apr. 1, 2011 (DE) 102011001743.7

(51) Int. Cl. C07K 1/14

(52) U.S. Cl. CPC C07K 1/14
USPC 530/415; 210/643

(57) ABSTRACT

A method to at least one of separate and purify biomolecules via an aqueous two-phase extraction includes providing the biomolecules in a first aqueous phase, providing a porous material comprising pores, providing a second aqueous phase at least in the pores of the porous material, and, at least one of separating and purifying the biomolecules by partially transferring the biomolecules from the first aqueous phase to the second aqueous phase. A dissolution of the biomolecules into the second aqueous phase in the pores of the porous material occurs during the at least one of separating and purifying.

### CROSS REFERENCE TO PRIOR APPLICATIONS

This application is a U.S. National Phase application under 35 U.S.C. §371 of International Application No. PCT/EP2012/055434, filed on Mar. 27, 2012 and which claims benefit to German Patent Application No. 10 2011 001 743.7, filed on Apr. 1, 2011. The International Application was published in German on Oct. 4, 2012 as WO 2012/130855 A1 under PCT Article 21(2).

### FIELD

The present invention relates to the field of separation and/or purification of biomolecules, particularly to the field of separation and/or purification of biomolecules by means of an aqueous two-phase extraction.

### BACKGROUND

Aqueous two-phase extraction (ATPE) is a widespread separation method in the field of analysis and production of biomolecules but has hitherto not been used on a large scale. This lies essentially in the often only very slow phase separation of the two aqueous phases and also in the frequent phenomenon that, as a result of the presence of the biomolecules, persistent emulsions form which can only be separated by (strong) centrifugation.

### SUMMARY

The object is therefore to provide an improved two-phase extraction process.

In an embodiment, the present invention provides a method to at least one of separate and purify biomolecules via an aqueous two-phase extraction which includes providing the biomolecules in a first aqueous phase, providing a porous material comprising pores, providing a second aqueous phase at least in the pores of the porous material, and, at least one of separating and purifying the biomolecules by partially transferring the biomolecules from the first aqueous phase to the second aqueous phase. A dissolution of the biomolecules into the second aqueous phase in the pores of the porous material occurs during the at least one of separating and purifying.

### DETAILED DESCRIPTION

The term “separation” is in particular understood as meaning all processes in which:
biomolecules are isolated from fermentation broths in pure form by dissolution in the second aqueous phase (capture),
groups of two or more of the aforementioned biomolecules are separated off from the fermentation medium,
biomolecules are isolated in pure form from the liquid media produced during biocatalysis (capture),
groups of two or more of the aforementioned biomolecules are separated off from biocatalysis media,
undesired secondary components, such as, for example, all types of undesired proteins (proteases, membrane proteins, etc.), viruses, DNA, cell organelles (mitochondria, etc.) and other cell debris, lipids, metabolites and metabolic products are removed individually or in groups from fermentation broths,
undesired secondary components are removed individually or in groups from the liquid media produced during biocatalysis.

The term “purification” is understood in particular as meaning all processes in which:
biomolecules are isolated from fermentation broths in pure form by dissolution in the second aqueous phase (polishing),
biomolecules are isolated in pure form from the liquid media produced during biocatalysis by dissolution in the second aqueous phase (polishing).

Within the context of the present invention, the term “biomolecules” is understood to mean (but is not limited to) all naturally occurring or artificially introduced molecules in biological samples. The term “biomolecules” is in particular understood as meaning nucleic acids, antibiotics, amino acids, lipids, carbohydrates, metabolites, inclusion bodies, metabolic products and in particular all types of proteins, including (but not limited to):
transport proteins,
antibodies,
membrane proteins,
hormones,
blood clotting factors,
enzymes,
collagens,
endotoxins.

The term “porous material” is in particular understood as meaning, but is not limited to, all materials which have an open porosity of 50%.

It has surprisingly been found that an aqueous two-phase extraction can be significantly improved by means of such a porous material. For many applications within the present invention, the process according to the present invention satisfies at least one or more of the following advantages:
As a result of the fact that the second aqueous phase is present in the porous material, a separation off of the biomolecules which stay in this aqueous phase (and thus “within” the porous material) after separation has taken place can take place in a simple manner.
Avoiding laborious phase separation results in a considerable time saving; it is possible to dispense with usually necessary separate centrifugation steps and with the expenditure on apparatus associated therewith.
The space requirement compared to aqueous two-phase extraction is reduced since the centrifugation step is spared; besides the saving in space, this results in a saving in investment costs.
A simple scale-up by using the heuristics and calculation model known in adsorption and chromatography.

In an embodiment of the present invention, the average pore size of the porous material can, for example, be from 0.1 nm to 5000 nm. This has been tried and tested in practice. The average pore size of the porous material can, for example, be from 2 nm to 2000 nm, for example, from 50 nm to 1000 nm.

The term “average” here means in particular that it is the mathematically averaged value of the entire diameters of the pores scattered (usually) according to a Gaussian distribution over a particle or a particle charge depending on production. Since the determination of the pore diameters is often carried out with the help of imaging processes, for the purposes of the present invention, pore diameter is understood in particular as meaning the diameter of the pores visible on the particle surface.

As has been shown in practice for many applications, that a high porosity signifies, on account of large constant pore diameters and/or pores that expand into the inside of particles and/or a central hollow cavity present in the particle, a high capacity for the absorption of second aqueous phase and thus potentially a higher capacity for the target component(s) to be separated off than compared to particles with a lower constant pore diameter and/or pores whose cross section becomes narrower into the inside of the particle, and/or without central hollow cavity.

In an embodiment of the present invention, while carrying out the separation and/or purification, the second aqueous phase can, for example, essentially be present in the pores of the porous material.

Within the context of the present invention, “essentially” here means 90% by weight, for example, 95% by weight, for example, 97% by weight, and, for example, 99% by weight.

By carrying out the separation in this way, the biomolecules located in the second aqueous phase also stay within the porous material. The present process is not, however, limited thereto. Depending on the specific embodiment, it may also be advantageous for the second aqueous phase to also be located outside of the porous material, it being used, so to speak, more “as support”.

In an embodiment of the present invention, before and/or after the separation, the first aqueous phase can, for example, be located essentially outside of the porous material. The separation of the phases is thereby facilitated.

In an embodiment of the present invention, the porous material can, for example, essentially be a particulate material. One or more of the following advantages can often be achieved thereby:
The particles can be packed in a column and thus be used as a preparative “quasi-chromatographic” fixed bed.
Since the pores of the particles serve as “hollow cavity” for the second aqueous phase, the particles can easily be reused, and moreover, rapid adaptation/adjustment is possible, as well as a GMP-conforming (re)impregnation in sealed apparatuses.

In an embodiment of the present invention, the average particle size of the porous material can, for example, be from 0.01 mm and 20 mm, for example, 0.05 mm and 15 mm, for example, 0.1 mm and 5 mm, and, for example, 2 mm.

In an embodiment of the present invention, during the separation, an adsorption of biomolecules onto the porous material can, for example, take place. This is often a welcome “secondary effect” when using certain porous materials.

In an embodiment of the present invention, the porous material can, for example, have a swelling rate of 40%. This has proven to be advantageous in practice since in this way no “leaking” of the second aqueous phase located in the porous material takes place. For many applications, however, a certain swelling rate is advantageous since the capacity for the second aqueous phase thereby increases. In an embodiment of the present invention, the porous material can, for example, have a swelling rate of 20% and 35%.

In an embodiment of the present invention, the porous material can, for example, essentially be selected from the group comprising:
polymer particles based on polystyrene-divinylbenzene, polypropylene, melamine, poly(methyl methacrylate), polyvinyl acetate, polyvinyl chloride, polystyrene, polyacrylamide, polybutyl acrylate; these copolymers optionally in amorphous, semicrystalline and crystalline form,
magnetic (polymer) particles,
microcapsules,
carbon-containing polymer particles, so-called carbonaceous resins,
coated polymer particles, so-called coated particles, where the coating can be achieved inter alia by polyvinyl alcohol,
polymeric anionic and cationic exchangers,
zeolites,
silica/silicates,
activated carbon,
inorganic anionic and cationic exchangers,
metal organic frameworks,
glass,
carbonates,
polymer foams,
metal foams,

or mixtures thereof.

In an embodiment of the present invention, the aqueous two-phase extraction used can, for example, be a polymeric two-phase extraction (ATPPE), a micellar two-phase extraction (ATPME), a reverse micellar two-phase extraction (ATPRME) and/or a two-phase extraction using ionic liquids.

The individual extraction processes are discussed in more detail below.

### Polymeric Two-Phase Extraction (ATPPE)

In an embodiment of the present invention, the following specifications can, for example, be used if a polymeric two-phase extraction (ATPPE) is used:

The following systems can, for example, be used if the first and second aqueous phase comprise nonionic polymers:
polyoxy compounds such as polyethylene glycol/polypropylene glycol on the one hand, polysaccharides such as, for example, dextran on the other hand,
polyoxy compounds such as polyethylene glycol/polypropylene glycol on the one hand, polyhydroxy polymers such as polyvinyl alcohol on the other hand.

The following systems can, for example, be used if the first and second aqueous phase comprise a nonionic polymer and a polyelectrolyte:
polyoxy compounds such as polyethylene glycol/polypropylene glycol on the one hand, sulfonated polysaccharides such as, for example, sodium dextran sulfate on the other hand,
polyoxy compounds such as polyethylene glycol/polypropylene glycol on the one hand, polyimines such as polyethyleneimine on the other hand,
modified polysaccharides such as methylcellulose on the one hand, sulfonated polysaccharides such as, for example, sodium dextran sulfate on the other hand.

The following systems can, for example, be used if the first and second aqueous phase comprise electrolytes:
sulfonated polysaccharides such as, for example, sodium dextran sulfate on the one hand, modified polysaccharides such as sodium carboxydextran on the other hand,
modified polysaccharides such as sodium carboxycellulose on the one hand, differently modified polysaccharides such as sodium carboxydextran on the other hand.

The following systems can, for example, be used if the first and second aqueous phase comprise a nonionic polymer and a highly concentrated solution of a low molecular weight substance:
polyoxy compounds such as polyethylene glycol/polypropylene glycol on the one hand, low molecular weight saccharides such as glucose on the other hand,
polyoxy compounds such as polyethylene glycol/polypropylene glycol on the one hand, low molecular weight salts such as citrate or phosphate on the other hand.

To further improve the separation efficiency, the aforementioned polymers and polyelectrolytes can be coupled with affinity ligands, for example, glutaric-acid-, amino-, mercaptoethylpyridine-, pyrimidine-, benzyl- and triazine-based groups.

### Micellar Two-Phase Extraction (ATPME)

In an embodiment of the present invention, the following specifications can, for example, be used if micellar two-phase extraction (ATPME) is used:

The following systems can, for example, be used if the system contains nonionic surfactants:
an aqueous solution consisting of polyethylene-oxide-based nonionic surfactants such as n-decyltetraethylene oxide (C10E4) and octylphenol ethoxylate (Triton X-114), which, depending on the temperature, can be separated into a micelle-rich phase and a low-micelle phase.

The following systems can, for example, be used if the system contains nonionic and cationic surfactants:
an aqueous solution consisting of polyethylene-oxide-based nonionic surfactants such as n-decyltetraethylene oxide (C10E4) and cationic surfactants such as alkyltrimethylammonium bromide which, depending on the temperature, can be separated into a micelle-rich phase and a low-micelle phase.

The following systems can, for example, be used if the system contains a mixture of anionic and cationic surfactants:
an aqueous solution consisting of anionic surfactants such as dodecyltriammonium bromide (C12NE) and cationic surfactants such as sodium perfluorooctanoate (SPFO).

### Reverse Micellar Two-Phase Extraction (ATPRME)

In an embodiment of the present invention, the following specifications can, for example, be used if reverse micellar two-phase extraction (ATPRME) is used:

The following systems can, for example, be used if thefirst phase contains cationic surfactants and second phase contains organic solvents:
n-benzyl-n-dodecyl-n-bis(2-hydroxyethyl)ammonium chloride (BDBAC)/isooctane/hexanol,
cetyltrimethylammonium bromide (CTAB)/isooctane/hexanol/butanol.

Two-Phase Extraction Using Ionic Liquids

In an embodiment of the present invention, the following specifications can, for example, be used if the two-phase extraction with the use of ionic liquids is used:

The following systems can, for example, be used if the first aqueous phase contains ionic liquid and second aqueous phase contains electrolytes:
ionic liquids such as Ammoeng™ 110 on the one hand, salts such as potassium hydrogenphosphate (K2HPO4/KH2PO4) on the other hand,
ionic liquids such as 1-butyl-3-methylimidazolium chloride ([Bmim][Cl]), salts such as potassium hydrogenphosphate (K2HPO4) on the other hand,
ionic liquids such as 1-butyl-3-methylimidazolium tetrafluoroborate ([Bmim][BF4]), salts such as sodium hydrogenphosphate (NaH2PO4) on the other hand.

In an embodiment of the present invention, the process according to the present invention for the separation and/or purification of biomolecules can, for example, include the following steps:
a) “filling” or “impregnating” the porous material with the second aqueous phase,
b) suspending the porous material in the first aqueous phase,
c) adding the mixture containing the biomolecules to be separated and/or purified,
d) carrying out the separation,
e) separating off the porous material,
f) isolating the separated-off biomolecules from the second aqueous phase present in the pores of the porous material.

In an embodiment of the present invention, the second aqueous phase can, for example, be saturated with the first aqueous phase before or during step a). This has the advantage that during the separation no (or only a very small amount of) first aqueous phase penetrates into the pores of the porous material.

It is reasonable for the person skilled in the art that steps b) and c) can take place in any desired order or simultaneously. In some embodiments of the present invention, the separating off of the desired biomolecules also takes place directly from the “reaction solution” (for example, in the case of fermenter processes), i.e., the first aqueous phase and the mixture containing the biomolecules to be separated and/or purified are (partly) identical.

Step d) can take place either by merely waiting (i.e., by means of diffusion), but in most cases active thorough mixing will take place, this being able to be accomplished, for example, by stirring, for example, by means of an impeller, by streaming in a fixed bed similar to chromatographic methods or by dispersing in a fluidized bed by means of passing through a loose bed.

Step e) can be accomplished in the simplest case by filtering off the porous material.

The filtration can be achieved by dynamic filtration (for example, crossflow filtration) and also by static filtration (for example, pressure or membrane filtration).

When using a magnetic porous material, the separating off of the porous material can be achieved by applying an (electro)magnetic field. The separating off of the porous material can furthermore be achieved by gravimetric settling. A centrifuge or a decanter can moreover be used to separate off the porous material.

Step f) can take place, for example, by back-extraction of the biomolecules from the second phase by means of concentrated salt solutions and/or by changing the process parameters, such as, for example, pH, ionic strength, addition of an additional salt.

A further embodiment of the present invention relates to a use of the process according to the present invention for:
the industrial production of proteins,
the industrial production of antibiotics, amino acids, (oligo)peptides, nucleic acids, lipids, carbohydrates, metabolites, metabolic products, inclusion bodies, plasmids,
the analytical separation of biomolecules,
the industrial removal of undesired components such as viruses, proteases, cell debris, etc. from media containing biomolecules for the purposes of precleaning the media containing biomolecules,
phytochemistry.

The aforementioned components and also the claimed components described in the embodiments and to be used according to the present invention are not subject to any particular exclusion conditions as regards their size, design, choice of material and technical conception, meaning that the selection criteria known in the field of application can be used without restriction.

Further details, features and advantages of the subject matter of the present invention arise from the dependent claims and also from the descriptions below of the appertaining examples, which are to be considered purely illustrative.

### Example I

### Separating Off of BSA Protein

The following specifications were chosen:
first aqueous phase: 20% w/w trisodium citrate solution
second aqueous phase: 20% (w/w) polyethylene glycol solution
porous material: Amberlite XAD16 particles (crosslinked polystyrene-divinylbenzene, porosity 0.55, pore size 10 nm, particle size 0.56-0.71 mm)
biomolecules to be separated off/purified: BSA (bovine serum albumin) protein

### a) “Filling” or Impregnation of the Porous Material.

For this purpose, essentially the method described by J. L. Cortina and A. Warshawsky, Developments in solid-liquid extraction by solvent-impregnated-resins, Ion Exch. Solvent Extr. 13, 195-293, 1997, was used. Firstly, the second aqueous phase was preequilibrated with first aqueous phase. Then, 2 g of porous material were dispersed completely in the second aqueous phase. The dispersion was placed into an ultrasound bath for a period of 30 min, then filtered, and the remains of aqueous phase adhering to the particles were removed with cellulose.

### b) Separating Off the Biomolecules

In the second step, the first aqueous phase was admixed with BSA protein solution, giving a concentration of 0.5 mg/ml of protein. Then, 2 g of the porous material were treated with 10 ml of first aqueous phase and dispersed for 180 min by shaking using an overhead shaker. Centrifugation was then carried out. A sample was taken from the supernatant and the remaining protein concentration was determined.

### c) Comparative Experiments

In parallel, a two-phase extraction was carried out without the addition of porous material. For this, 5 ml of the preequilibrated second phase were admixed with 10 ml of the protein-containing first phase and emulsified for 180 min with stirring. After phase separation had taken place, the remaining protein concentration in the first aqueous phase was determined.

As a further comparative experiment, the adsorption of the protein onto the porous material was determined. For this, a 0.5 mg/ml protein solution was admixed with porous material. The mixture was left to disperse (analogously to step b), the remaining protein concentration was then determined.

The experimental results are listed in Table I:

TABLE I

| | Depletion of |
|---|---|
| Method | the Protein (in %) |
| Method according to the present invention | 22.22% |
| Two-phase extraction without porous | 12.26% |
| material | |
| Adsorption from water | 8.65% |

It can thus clearly be seen that, as well as being able to carry out the process according to the present invention more quickly, the depletion of the protein was also considerably increased.

### d) Investigation of the Porous Material

In order to establish the degree of adsorption of the protein onto the porous material (in contrast to the solution in the second aqueous phase located in the pores), the following investigation was carried out.

The porous material containing the second phase (PEG solution) was separated by means of a vacuum pump and filter paper from the supernatant of the first phase (salt solution) containing the target protein. Then, 20 ml of the second phase (PEG solution) presaturated with the first phase (salt solution) were added to the porous material. This dispersion was mixed for 180 min in an overhead shaker at 10 rpm. Afterwards, the supernatant of the PEG solution surrounding the porous material was removed by means of a pipette, centrifuged (5000 rpm) and sterile-filtered. 5 ml of the filtered PEG solution were emulsified with 5 ml of presaturated (with PEG solution) salt solution for 180 min in an overhead shaker for the purposes of back-extracting the protein. The phases were then separated by centrifugation (1500 rpm) and a sample of the salt solution was taken. This was investigated as to its protein content by means of a UV/VIS spectrophotometer at 280 nm. These measurements revealed that at least circa 50% of the protein is present in dissolved form, i.e., not adsorbed, within the second phase present in the pores of the porous material.

### Example II

The following specifications were chosen:
first aqueous phase: 20% w/w dipotassiumhydrogenphosphate solution
second aqueous phase: 20% (w/w) polyethylene glycol solution
porous material: Amberlite XAD16 particles
biomolecules to be separated off/purified: BSA (bovine serum albumin) protein

The procedure corresponded to Example I. The experimental results are listed in Table II:

TABLE II

| | Depletion of |
|---|---|
| Method | the Protein (in %) |
| Method according to the present invention | 32.03% |
| Two-phase extraction without porous | 15.48% |
| material | |
| Adsorption from water | 8.65% |

### Examples III to V

The specifications of Examples III and V correspond to those from Example I, except that in each case a different porous material was chosen. The experimental results are listed in Table III.

TABLE III

| | Depletion of |
|---|---|
| Example | the Protein (in %) |
| Example III: Lewatit VPOC 1064 | 42.55% |
| Example IV: Amberlite XAD18 | 48.85% |
| Example V: Lewatit CNP 105 | 95.11% |

The properties of the porous materials used are summarized in Table IV.

TABLE IV

| | | | | Particle |
|---|---|---|---|---|
| | Chemical | Pore Size | Pore Volume | Diameter |
| Material | Composition | (in nm) | (in mg/L) | (in mm) |
| Lewatit | Crosslinked | 5-10 | 1.02 | 0.44-0.54 |
| VPOC | polystyrene | | | |
| 1064 | | | | |
| Amberlite | Crosslinked | 15 | n.s. | 0.375-0.475 |
| XAD18 | polystyrene- | | | |
| | divinylbenzene | | | |
| Lewatit | Methacrylate | ~27 | n.s. | 0.1-0.4 |
| CNP 105 | polymer | | | |

### Examples VI to XI

The specifications and results are summarized in Table V and VI

TABLE V

| Example | Protein | Two-Phase System | Particle |
|---|---|---|---|
| VI | Lysozyme | PEG2000/Na citrate | Amberlite |
| | | (20 wt %/20 wt %) | XAD16 |
| VII | Myoglobin | PEG2000/Na citrate | Amberlite |
| | | (20 wt %/20 wt %) | XAD16 |
| VIII | Bovine Serum | PEG2000/Na citrate | Amberlite |
| | Albumin | (20 wt %/20 wt %) | XAD16 |
| IX | Alpha- | PEG2000/Na citrate | Amberlite |
| | lactalbumin | (20 wt %/20 wt %) | XAD16 |
| X | Cytochrome C | PEG2000/Na citrate | Amberlite |
| | | (20 wt %/20 wt %) | XAD16 |
| XI | Bovine Serum | PEG4000/Dx500000 | Stuttgarter Masse |
| | Albumin | (Dextran) (8 wt %/12 wt %) | 3-5 (Pall) |

TABLE VI

| | Depletion of the protein | Depletion of the Protein | |
|---|---|---|---|
| | (Two-Phase Extraction) | (Method According to the | |
| Example | in % | Present Invention) in % | Factor |
| VI | 32.47 | 86.80 | 2.67 |
| VII | 12.53 | 66.73 | 5.33 |
| VIII | 9.63 | 41.69 | 4.33 |
| IX | 6.10 | 12.86 | 2.11 |
| X | 3.08 | 6.37 | 2.06 |
| XI | 0.63 | 0.73 | 1.16 |

The individual combinations of the constituents and of the features of the variants already mentioned are exemplary; the replacement and the substitution of these teachings with other teachings contained in this document with the cited documents are likewise expressly contemplated. The person skilled in the art is aware that variations, modifications and other explanations which are described here may likewise arise without deviating from the inventive concept and the scope of the present invention. The description given above is accordingly to be considered as an example and non-limiting. The word include used in the claims does not exclude other constituents or steps. The indefinite article “a” does not exclude the meaning of a plural. The mere fact that certain measures are recited in mutually different claims does not mean that a combination of these features cannot be used to advantage. The scope of the present invention is defined in the claims which follow and the associated equivalents.

What is claimed is:

1-10. (canceled)

11. A method to at least one of separate and purify biomolecules via an aqueous two-phase extraction, the method comprising:
providing the biomolecules in a first aqueous phase;
providing a porous material comprising pores;
providing a second aqueous phase at least in the pores of the porous material; and
at least one of separating and purifying the biomolecules by partially transferring the biomolecules from the first aqueous phase to the second aqueous phase, wherein, during the at least one of separating and purifying, a dissolution of the biomolecules into the second aqueous phase in the pores of the porous material occurs.

12. The process as recited in claim 11, wherein an average pore size of the pores of the porous material is from 0.1 nm to 5,000 nm.

13. The process as recited in claim 11, wherein the second aqueous phase is essentially present only in the pores of the porous material during the at least one of separating and purifying.

14. The process as recited in claim 11, wherein the first aqueous phase is essentially present only outside of the pores of the porous material at least one of before and after the at least one of separating and purifying.

15. The process as recited in claim 11, wherein the porous material is a particulate material.

16. The process as recited in claim 11, wherein an average particle size of the porous material is 0.01 mm and 20 mm.

17. The process as recited in claim 11, wherein, during the at least one of separating and purifying, an adsorption of the biomolecules onto the porous material occurs.

18. The process as recited in claim 11, wherein the porous material has a swelling rate of 40%.

19. The process as recited in claim 11, wherein the porous material is essentially selected from the group comprising:
polymer particles based on polystyrene-divinylbenzene, polypropylene, melamine, poly(methyl methacrylate), polyvinyl acetate, polyvinyl chloride, polystyrene, polyacrylamide, polybutyl acrylate,
magnetic (polymer) particles,
microcapsules,
carbon-containing polymer particles (so-called carbonaceous resins),
coated polymer particles (so-called coated particles),
polymeric anionic and cationic exchangers,
zeolites,
silica,
silicates,
activated carbon,
inorganic anionic exchangers,
inorganic cationic exchangers,
metal organic frameworks,
glass,
carbonates,
polymer foams, and
metal foams,
or mixtures thereof.

20. The process as recited in claim 19, wherein,
the polymer particles based on polystyrene-divinylbenzene, polypropylene, melamine, poly(methyl methacrylate), polyvinyl acetate, polyvinyl chloride, polystyrene, polyacrylamide, polybutyl acrylate are provided in at least one of an amorphous, a semi-crystalline, and a crystalline form, and
a coating of the coated polymer particles (so-called coated particles) are obtained by polyvinyl alcohol.

21. A method of using the method as recited in claim 11 for at least one of an industrial production of at least one of proteins, antibiotics, amino acids, (oligo)peptides, nucleic acids, lipids, carbohydrates, metabolites, metabolic products, inclusion bodies, and plasmids, an analytical separation of biomolecules, an industrial removal of undesired components from media containing biomolecules to preclean the media containing biomolecules, and phytochemistry, the method comprising:
providing at least one of a protein, an antibiotic, an amino acid, an (oligo)peptide, a nucleic acid, a lipid, a carbohydrate, a metabolite, a metabolic product, an inclusion body, a plasmid, an undesired component in a media, and a phytochemical, which each respectively contain biomolecules, in a first aqueous phase;
providing a porous material comprising pores;
providing a second aqueous phase at least in the pores of the porous material; and
at least one of separating and purifying the biomolecules in the at least one of the protein, the antibiotic, the amino acid, the (oligo)peptide, the nucleic acid, the lipid, the carbohydrate, the metabolite, the metabolic product, the inclusion body, the plasmid, the undesired component in the media, and the phytochemical by partially transferring the biomolecules from the first aqueous phase to the second aqueous phase, wherein, during the at least one of separating and purifying, a dissolution of the biomolecules into the second aqueous phase in the pores of the porous material occurs.

22. The method as recited in claim 21, wherein the undesired component includes viruses, proteases, and cell debris.
